(19) United States
(12) Patent Application Publication
(10) Pub. No.: US 2022/0215911 A1
(43) Pub. Date: Jul. 7, 2022

(54) PATIENT MANAGEMENT SYSTEM

(71) Applicant: Drägerwerk AG & Co. KGaA, Lübeck (DE)

(72) Inventors: Götz KULLIK, Lübeck (DE); Frank FRANZ, Lübeck (DE); Birger LANDWEHR, Lübeck (DE)

(21) Appl. No.: 17/568,291

(22) Filed: Jan. 4, 2022

(30) Foreign Application Priority Data
Jan. 5, 2021 (DE) 102021100062.9

(51) Int. Cl. G16H 10/60; G16H 20/10

(52) U.S. Cl. CPC G16H 10/60; G16H 20/10

(57) ABSTRACT

A patient management system (100) for managing an assignment to a patient, includes a plurality of patient identifiers (110). The patient identifiers are intended for use by a medical staff. Each of the patient identifiers, in which patient-specific information (112) is stored, is assigned to a corresponding one of a plurality of patients. A mobile user carrier device (120) is configured to store a plurality of patient identifiers, so that a number of patient identifiers currently being stored at the mobile user carrier device can be visually recognized. The mobile user carrier device has an energy storage device (122) and a communication interface (124). The mobile user carrier device is configured to read out the respective patient-specific information of the patient identifiers being stored at the mobile user carrier device, at least partially, and to output same wirelessly via the communication interface.

### CROSS REFERENCE TO RELATED APPLICATIONS

This application claims the benefit of priority under 35 U.S.C. § 119 of German Application 10 2021 100 062.9, filed Jan. 5, 2021, the entire contents of which are incorporated herein by reference.

### TECHNICAL FIELD

The present invention pertains to a patient management system for managing an assignment to a patient. The present invention pertains, furthermore, to a process for managing an assignment to a patient.

### TECHNICAL BACKGROUND

It is common in a hospital setting to assign the responsibility for certain patients to a medical professional staff for the duration of a work shift. For example, a number of patients are assigned to each nursing staff member at the beginning of the service. In order to ensure that at least one medical professional, e.g., a nursing staff member, is assigned to each patient, the current assignment may be stored, for example, in a management system.

It is known from DE 102017010149 A1 for the transfer of the responsibility for a patient at the time of a change of shifts that a mobile device assigned to a first nursing staff member communicates for this purpose with a mobile device assigned to a second nursing staff member. The patient transfer is started by one of these devices and the second mobile device receives the assignment, the assignment information proper running in the background over a server, which stores the assignment information. Change of the assignment can be indicated, for example, by these two devices being brought together in space.

### SUMMARY

An object of the present invention is to provide an improved patient management system, and in particular an especially transparent and especially reliable patient management system.

A patient management system for management and assignment to a patient, with a plurality of patient identifiers and with at least one central patient monitoring device is proposed according to the present invention for accomplishing this object.

A patient identifier intended for a medical staff (nursing staff) member, in which patient-specific information for a particular specific patient is stored, is assigned to a corresponding plurality of patients.

The at least one mobile user carrier device is configured to store (hold and carry) a plurality of patient identifiers, especially to store them in an visually recognizable (optically detectable-viewable-optically discernible) manner, so that a number of patient identifiers being stored in the mobile user carrier device are optically detectable, the mobile user carrier device having an energy storage device and a communication interface, and the mobile user carrier device being configured partially to read the respective patient-specific information of the patient identifiers being stored in the mobile user carrier device and to output them wirelessly (in a wireless manner) via the communication interface, especially at recurring time intervals.

It was found within the framework of the present invention that it cannot be detected in a clinical setting for a specific nursing staff member how many patients are assigned to this nursing staff member, so that the current load of this nursing staff manner can hardly be taken into consideration at the time of the distribution of acute tasks. In addition, it was found that the assignment of patient and nursing staff member must be carried out so reliably that it is always detected immediately when no currently present nursing staff member is being assigned to a patient. The wearing of a patient identifier assigned to a specific patient by the nursing staff member is provided according to the present invention to solve this problem.

Due to the number of patient identifiers currently being stored in the mobile user carrier device being viewable (visually recognizable), it is always possible to detect optically whether the appropriate number of patients are assigned to a nursing staff member or not. As a result, it can be avoided that additional tasks are assigned to a nursing staff member to whom very many patients have been assigned, so that this person may become overtaxed.

A nurse or health care staff member is defined within the framework of the present invention as a person who helps a patient during recovery. He or she may be a nursing staff member, for example, a nurse, or a physician or the like.

The output of least some of the patient-specific information being stored by the mobile user carrier device makes it possible to ensure in an automated manner that at least one nursing staff member is assigned to each patient. In particular, it can also be ensured by the communication with the mobile user carrier device that the assigned nursing staff member is present within the environment of the hospital or of another treatment area when she is carrying the mobile user carrier device.

The patient-specific information is a character string, which can be unambiguously assigned to the patient, e.g., a name, a registration number, a hospital bed number, an image or the like and/or a combination thereof. The character string must be suitable for inferring on the basis of the character string of the patient currently being assigned to this character string.

The storage of the patient identifiers at the mobile user carrier device may take place, for example, via a suitable storage mechanism, e.g., an identifier receptacle, and/or via a magnetic interaction between the patient identifier and the mobile user carrier device. The number of currently assigned patient identifiers is preferably possible by a direct detection of the corresponding patient identifiers. The mobile user carrier device especially preferably has a plurality of identifier receptacles, via which the number of patient identifiers currently being stored at the mobile user carrier device are stored in a viewable manner. As an alternative or in addition, the mobile user carrier device of a currently assigned number of patient identifiers can display a currently assigned number of patient identifiers in a viewable, possibly without the patient identifier itself being able to be seen.

According to the present invention, a respective patient identifier is a physical object, which must be passed on physically at the time of a change in responsibility for a specific patient, for example, at the time of a change of shifts. It is possible thereby to always ensure, even in case of a great fluctuation of the responsibilities for a patient and/or of a great fluctuation of patients, that no patient will remain without an assigned nursing staff member. The patient identifier is assigned to the corresponding patient and is given to a nursing staff member preferably at the beginning of the treatment, for example, at the time of admission of the patient in the hospital, and the nursing staff member who is currently in possession of this patient identifier will always know from then on that she is responsible for this patient.

In addition, the transfer of the entire mobile user carrier device with all the assigned patient identifiers, which is made possible by the patient management system according to the present invention, is especially advantageous. For example, the responsibility for a group of patients can be transferred hereby rapidly and reliably by the transfer of the mobile user carrier device in case of a temporary absence and/or at the time of a change of shifts.

The communication interface of the mobile user carrier device advantageously makes it possible to automatically detect the patient assigned to this mobile user carrier device by devices that are configured to communicate with the corresponding communication interface.

The mobile user carrier device is preferably an object that is readily detectable from the outside, e.g., a necklace, a bracelet or a clip.

The use of the patient management system being described is especially advantageous in the care of patients in an intensive care unit. The mobile user carrier device is preferably configured against this background to store at most 10 patient identifiers, especially at most 8 patient identifiers, and especially preferably at most 6 patient identifiers in a viewable manner. The number of patient identifiers that can be assigned as a maximum is preferably limited in this manner because the care for patients being treated in intensive care units is especially time-consuming.

A plurality of patient identifiers may be assigned according to the present invention to a single patient. This may happen, for example, when both a nursing staff member and a physician carry a corresponding patient identifier for this patient in their respective mobile user carrier devices. It can be ensured hereby, for example, after a surgery that an attending physician is also informed of the current state of the patient in addition to a nursing staff member.

Preferred embodiments of the patient management system according to the present invention will be described below.

In a preferred embodiment, each patient identifier from the plurality of patient identifiers has an internal memory, which is configured to store the patient-specific information at least for the duration of the treatment of the corresponding patient. This memory is preferably filled with the patient-specific information at the beginning of the treatment of a patient, for example, at the time of admission to the hospital. As an alternative, the patient identifier may always have the same patient-specific information, which is assigned to the patient at the beginning of the treatment of a patient and it is as a result patient-specific information for this patient. Consequently, patients who are not being treated at the same time in the hospital but to whom the same patient identifier has been assigned would also have the same patient-specific information, namely, the information assigned to the patient identifier.

In another, especially advantageous embodiment, each patient identifier from the plurality of patient identifiers has an identifier interface, which is configured for the wireless transmission of the patient-specific information. The reading of at least some of the patient-specific information by the mobile user carrier device is thus carried out via this identifier interface. The identifier interface is preferably configured such that each or at least almost each mobile user carrier device of the patient management system according to the present invention can read at least partially the corresponding patient-specific information via the identifier interface. In an especially preferred variant of this embodiment, the identifier interface is a near-field communication (NFC) interface or a radio-frequency identification (RFID) interface for the wireless transmission. The use of such a prior-art technology makes possible a favorable manufacture of the corresponding patient identifiers by purchasing prior-art NFC and/or RFID components. In addition, the use of such widespread technologies makes it possible to provide the patient management system according to the present invention in an especially reliable manner. As an alternative or in addition, a patient identifier may have a QR code (Quick Response code) or the like, so that the reading by the mobile user carrier device is carried out via an optical reading of the QR code or the like.

The mobile user carrier device is configured in an advantageous embodiment to receive a patient signal assigned to the patient identifier from a managing device and to trigger a corresponding output of the mobile user carrier device and/or of the corresponding patient identifier. The triggered output may pertain to a signal, which is outputted to an external device. Furthermore, the output may pertain to a direct output perceptible directly at the patient identifier and/or directly at the mobile user carrier device. In a preferred variant of this embodiment, the output takes place at least partially as a tactile output, as an optical output and/or as an acoustic output.

In an advantageous variant of the above embodiment or in an example of the above variant, the corresponding output of the mobile user carrier device and/or of the corresponding patient identifier comprises a wireless output to an external output device located in a predefined environment in space in order to trigger a device output, especially an alarm output or a treatment output, by the external output device. The combination of mobile user carrier device and patient identifier makes possible in this embodiment a direct communication with the external output device located in the area around it. For example, a communication may take place with a medical device, with a floor display, with a management center, with a break room display or the like. The person who is currently in possession of the mobile user carrier device with the corresponding patient identifier can thus advantageously be alerted to a critical state of the patient assigned to that person, to connection problems of that person's mobile user carrier device, to a treatment recommendation for the treatment of the patient assigned to that person or the like.

In an especially advantageous embodiment, the patient management system has, furthermore, a patient monitoring device, which is configured to detect and to store an assignment between the patient-specific information and the mobile user carrier device with the corresponding patient identifier on the basis of the output of the communication interface of the mobile user carrier device. It is possible as a result to automatically detect the mobile user carrier device to which the corresponding patient identifier is currently physically assigned. A manual effort needed for assigning the patients to the corresponding nursing staff is reduced hereby. Furthermore, it is possible via the patient monitoring device to monitor centrally that a nursing staff member is also always assigned to a respective patient. Finally, the patient monitoring device also makes possible a reliable documentation of which nursing staff member was assigned to which patient and when. Problems that may possibly arise in the assignment between patient and nursing staff member can be investigated as a result later and possibly existing sources of error in the assignment can be corrected.

The patient monitoring device is further configured in an especially preferred variant of the above embodiment to assign an authorization level to the respective mobile user carrier device and to carry out a communication, which is triggered by a patient identifier assigned to this mobile user carrier device, on the basis of the assigned authorization level. It is ensured in this variant that information to be possibly outputted via the communication is actually outputted only if the mobile user carrier device has the corresponding authorization level. It can thus be ensured that a nurse cannot see all the confidential information of the patient at a medical device, which would possibly be outputted for the mobile user carrier device of a physician. As an alternative or in addition, for example, an especially complex medical device cannot be switched on during the communication with the mobile user carrier device of a nursing staff member to change parameters, whereas the same medical device activates itself for changing parameters in case of communication with the mobile user carrier device of a physician.

In another embodiment, the patient management system has, furthermore, two locating units, via which a position of at least one mobile user carrier device in a region (building or campus) can be determined, wherein the patient monitoring device is configured to assign the correspondingly determined position in the region (building or campus) to the mobile user carrier device or to the patient-specific information. A current location of the assigned mobile user carrier device can especially advantageously be assigned to each patient in this embodiment via the assignment to the mobile user carrier device. For example, it can be decided hereby in case of an acute alarm whether the assigned mobile user carrier device is informed or whether a mobile user carrier device located markedly closer to the patient is informed about the patient to be treated. Furthermore, it can be determined by the use of the locating units whether the mobile user carrier device currently assigned to the patient is in the area of the hospital at all and possibly when it has left this area. For example, it can thus be detected whether the assigned nursing staff member is just having a break, for example, a smoking break, outside the hospital. The determination of a position by two locating units is carried out, for example, by measuring a signal intensity of an output of the respective mobile user carrier device by both locating units, so that a location within the hospital can be determined on the basis of a distance between the two locating units. As an alternative or in addition, a location can be determined by means of a third locating unit by triangulation of the corresponding signals in the known manner. The use of such locating units is known to the person skilled in the art and will not therefore be explained in detail below.

The patient monitoring device is configured in an advantageous variant of the above embodiment to trigger a device output, especially an alarm generation output or a treatment output, by the external output device depending on the assigned position at an external output device located in an area surrounding this assigned position. The fact that the position of the mobile user carrier device is known via the at least two locating units is advantageously utilized in this variant. An external output device in the area of this position can thus be actuated directly to output information relevant for the user of the mobile user carrier device, especially information concerning the status of the assigned patient in a detectable manner.

The patient monitoring device is preferably connected to an alarm system of the corresponding institution, for example, the hospital, so that an alarm generation triggered by the alarm system can be displayed to the user of the mobile user carrier device via the patient management system according to the present invention, especially via an external output device and/or via the mobile user carrier device. The display is preferably carried out by an optical indicator, e.g., a display output and/or an LED of the external output device, of the mobile user carrier device and/or of the corresponding patient identifier.

In an advantageous embodiment, the patient monitoring device is further configured to receive a forwarded signal assigned to the patient identifier, especially from a medical device intended for monitoring the corresponding patient, and to output the forwarded signal as a patient signal to the mobile user carrier device. The mobile user carrier device is configured in this case to receive the patient signal, for example, via the communication interface and to trigger a corresponding output. As a result, an output in the environment of the user of the mobile user carrier device can be advantageously triggered directly by the medical device. It is possible hereby to call attention to a treatment result, to a treatment recommendation, to a change in the state of the patient, to a technical disturbance, to a change in the operating state of the medical device or the like.

The patient monitoring device is further configured in another advantageous embodiment to trigger a disconnection output if a communication channel to the mobile user carrier device is disconnected, if this mobile user carrier device is assigned to a patient identifier being monitored. It is advantageously ensured in this embodiment that in case of a technical defect or if an area being monitored is left, a disconnection output is generated in order to provide information showing that the nurse assigned to a patient is not probably available currently for the care of the patient. The patient monitoring device is preferably configured in this case, furthermore, to wait for a predefined time interval before an actually perceptible output is carried out after the disconnection output, which is then to be triggered. It is ensured thereby that a temporary disconnection problem or the fact that the area being monitored has been left for a short time does not lead to a disconnection output and does not lead to a corresponding management effort.

In another embodiment, the patient management system according to the present invention further has a plurality of patient-specific visitor identifiers, and the patient-specific information of the patient assigned to the visitor identifier is stored in each patient-specific visitor identifier. Such a visitor identifier makes it possible to automatically detect an authorization level for a person typically unknown to the hospital staff. For example, the blocking or unblocking of areas of the hospital can be brought about by means of the visitor identifier. It can thus be detected through a communication with the patient monitoring device by an external device to which patient the person with the corresponding visitor identifier is assigned and whether this person has an authorized interest in obtaining requested information, to open a requested door, to switch on a requested device, and/or to receive a requested service. The patient management system is advantageously combined in this embodiment with the management of visitor authorizations. A possible manual management effort is reduced hereby.

In an especially preferred embodiment, a respective patient identifier is configured such that it make possible an unambiguous optical identification (provides unambiguous optical identifiability) of the patient assigned to the patient identifier by means of a color, a symbol, a character string, indicia, markings or the like (indicia, markings, etc.). It is possible in this embodiment not only optically to detect the number of the patient identifiers currently assigned to the mobile user carrier device, but also to detect a specific patient or at least a patient classification of the corresponding patient, such as a patient in the intensive care unit, a comatose patient, and a patient in a postoperative state. The assignment of a color within the framework of this embodiment can correspond within the framework of this embodiment, for example, to a bed color assigned to the patient, to a door color of the hospital room or the like.

The energy storage device may be, for example, a replaceable battery or a rechargeable battery that can be charged in a contactless manner or the like. The energy storage device can especially preferably be charged after removal of the patient identifiers, for example, after a change of shifts.

According to another aspect of the present invention, a process for managing an assignment to a patient, which has the following steps, is proposed for accomplishing the above-described object:
Storage of patient-specific information in a patient identifier;
storage of a number of patient identifiers at a mobile user carrier device, so that a number of patient identifiers currently being stored in the mobile user carrier device can be visually recognized (viewed, optically detected, optically discerned), wherein the mobile user carrier device has an energy storage device and a communication interface;
reading out of the respective patient-specific information of the patient identifiers being stored in the mobile user carrier device by the mobile user carrier device; and
wireless output of the read-out patient-specific information via the communication interface.

The process according to the present invention is carried out by the patient management system according to the present invention and it thus has all the advantages described for this system. In particular, the process according to the present invention makes possible an especially reliable assignment between patient and nursing staff, because this assignment is embodied not only at the abstract level, for example, by a work instruction and/or by storing this assignment in a data bank, but at a physical level by the possession of the patient identifier for a specific patient by the nursing staff member. The transfer of the responsibility for patients can take place especially simply, rapidly and reliably especially at the time of a change of shifts.

The carrying out of the process described is typically preceded by a provision of the plurality of patient identifiers and of the at least one mobile user carrier device as a preceding step.

The process according to the present invention may also be expanded by one or more process steps corresponding to the advantageous embodiments described for the patient management system.

The additional steps of receiving a patient signal assigned to the patient identifier and of triggering a corresponding output in response to the patient signal are carried out in an advantageous embodiment.

In an alternative or additional embodiment, the process according to the present invention has, furthermore, a detection and storage of an assignment between the patient-specific information and the mobile user carrier device based on the output of the mobile user carrier device.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of a patient management system according to the present invention;

FIG. 2 is a schematic view of a second exemplary embodiment of the patient management system according to the present invention;

FIG. 3 is a schematic view of a third exemplary embodiment of the patient management system according to the present invention;

FIG. 4 is a schematic view of a fourth exemplary embodiment of the patient management system according to the present invention; and

FIG. 5 is a flow chart of an exemplary embodiment of a process according to another aspect of the present invention.

### DESCRIPTION OF PREFERRED EMBODIMENTS

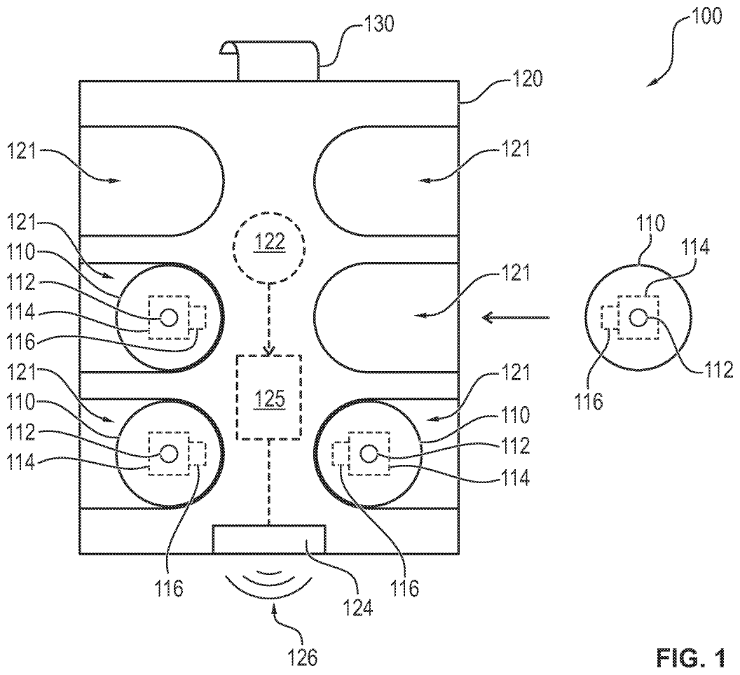

Referring to the drawings, FIG. 1 shows a schematic view of a first exemplary embodiment of a patient management system 100 according to the present invention.

The patient management system 100 according to the present invention is configured for managing an assignment to a patient and comprises a plurality of patient identifiers 100 and at least one mobile user carrier device 120. The assignment is an assignment between at least one nursing staff member and a patient.

A patient identifier 110 intended for the nursing staff, in which patient-specific information 112 is stored, is assigned to a corresponding patient of a plurality of patients. The respective patient identifier has for this purpose in the exemplary embodiment being shown an internal memory 114, which is configured to store the patient-specific information 112 for a duration of treatment of the corresponding patient. In the exemplary embodiment shown, the patient-specific information is a character string, which characterizes the patient, such as a name or a date of birth of the patient. Furthermore, the patient identifier has an identifier interface 116, which is configured for the wireless transmission of the patient-specific information 112. The identifier interface 116 is an RFID interface in this case. The identifier interface 116 is an NFC interface in an alternative exemplary embodiment.

The at least one mobile user carrier device 120 is configured to store a plurality of patient identifiers 110, especially in a viewable manner. A number of the patient identifiers 110 currently being stored in the mobile user carrier device 120 can thus be detected optically. The mobile user carrier device 120 may be arranged in the present exemplary embodiment by means of a clip 130 at the corresponding nursing staff member assigned to the mobile user carrier device 120. The mobile user carrier device 120 has an energy storage device 122 and a communication interface 124. The energy storage device 122 may be, for example, a replaceable battery or a rechargeable battery that can be charged in a contactless manner or the like. Furthermore, the mobile user carrier device 120 has a receiving unit 125, which is configured to receive at least some of the patient-specific information 112 via the identifier interface 116. Some of the patient-specific information may have, for example, parts of the name of the patient and/or parts of the date of birth or a combination of these data. It may be a passive RFID tag or an active RFID tag in this case. The parts of the patient-specific information read out via the receiving unit 125 are then outputted via the communication interface 124 wirelessly as an output signal. The output is carried out in this case at recurring time intervals, e.g., after less than 10 minutes, especially after less than 5 minutes, and especially preferably after less than 1 minute.

Three patient identifiers 110 are inserted in corresponding identifier receptacles 121 of the mobile user carrier device 120 in the exemplary embodiment shown. The insertion of a fourth patient identifier 110 is illustrated on the basis of the view of the corresponding insertion direction for the still free identifier receptacles 120. The identifier receptacles operates here via a positive-locking and/or non-positive connection, especially via a locking mechanism, not shown. As an alternative or in addition, a magnetic interaction between the mobile user carrier device 120 and the corresponding patient identifier 110 can make possible a permanent storage. The storage of the patient identifier 110 at the mobile user carrier device 120 is carried out according to the present invention such that a rapid severing of the connection between these two physical components is possible at any time.

In the exemplary embodiment shown, the mobile user carrier device 120 is configured to store at most six patient identifiers 110. In one exemplary embodiment, not shown, the mobile user carrier device is configured to store at most ten patient identifiers. The mobile user carrier device 110 can preferably be worn by means of the clip 130 such that the patient identifiers 110 can be seen from the outside, so that it can be immediately detected how many patients are assigned to one person. It can thus be estimated, in particular, whether many patients or rather only a small number of patients are assigned to this person.

In one exemplary embodiment, not shown, a character string, a symbol, a color and/or an image can be recognized on a respective patient identifier, as a result of which the specific patient, who is assigned to this patient identifier, can be recognized. For example, each hospital bed in an intensive care unit may have a color of its own, and the patient correspondingly assigned to this hospital bed receives a patient identifier assigned to that patient, which has the color of the bed. An assignment of the patient identifier currently stored to the patient would as a result be possible intuitively for a person who is an outsider.

The wireless communication according to the present invention with other devices can take place by means of WLAN, Bluetooth, BLE, ZigBee, LoRa or the like. Corresponding interfaces are known to the person skilled in the art and will not therefore be explained in detail below.

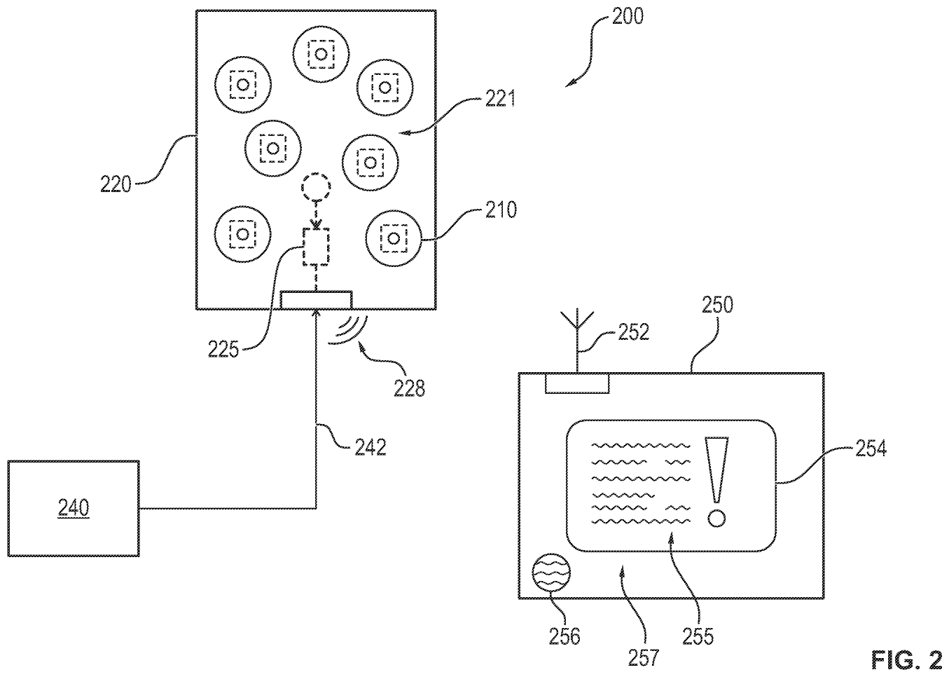

FIG. 2 shows a schematic view of a second exemplary embodiment of the patient management system 200 according to the present invention.

The patient management system 200 differs from the patient management system 100 shown in FIG. 1 in that the mobile user carrier device 220 is configured to receive a patient signal 242 assigned to the patient identifier 210 from a managing device 240. The receiving unit 225 of the mobile user carrier device 220 triggers via the patient signal 242 a corresponding output 228. The output 228 is received in the exemplary embodiment shown from an external output device 250, which is an external medical device in this case. The external output device 250 has an antenna unit 252 and a display 254 for this purpose. A visual output 255, which is triggered by the output 228 and the receipt thereof via the antenna unit 252, is outputted via the display 254. In addition, an acoustic signal is provided via a speaker 256 of the output device 250. The visual output 255 and the acoustic output, not shown, form the device output 257 of the external output device 250.

The patient signal 242 preferably comprises information, e.g., vital information of the patient or alarm information, which pertains to the patient. The mobile user carrier device 220 acts as an interface in order to provide the corresponding device output 257 precisely where the desired receiver of the corresponding information, namely, the user of the mobile user carrier device 220, is currently located. The device output 257 is preferably an alarm output or a treatment output.

In one exemplary embodiment, not shown, the output is a tactile output. This may be, for example, a vibration of the mobile user carrier device or of an additional mobile device connected to the mobile user carrier device, such as a smartphone, a pager or the like.

The managing device 240 may be connected to an alarm management system, so that the alarm management system triggers the patient signal 242 as an alarm output for the user of the mobile user carrier device 220 via the managing device 240.

Furthermore, the patient management system 200 differs from the patient management system 100 in that no identifier receptacles are provided at the mobile user carrier device 220, but the patient identifiers 210 are maintained (stored/carried) at the mobile user carrier device 220 in this case via a magnetic surface 221. The number of the patient identifiers 210 that can be assigned to the mobile user carrier device 220 is limited only by the size of the magnetic surface 221 rather than by the number of identifier receptacles.

The manner of functioning of the mobile user carrier device 220 is otherwise largely identical to that of the mobile user carrier device 120 from FIG. 1.

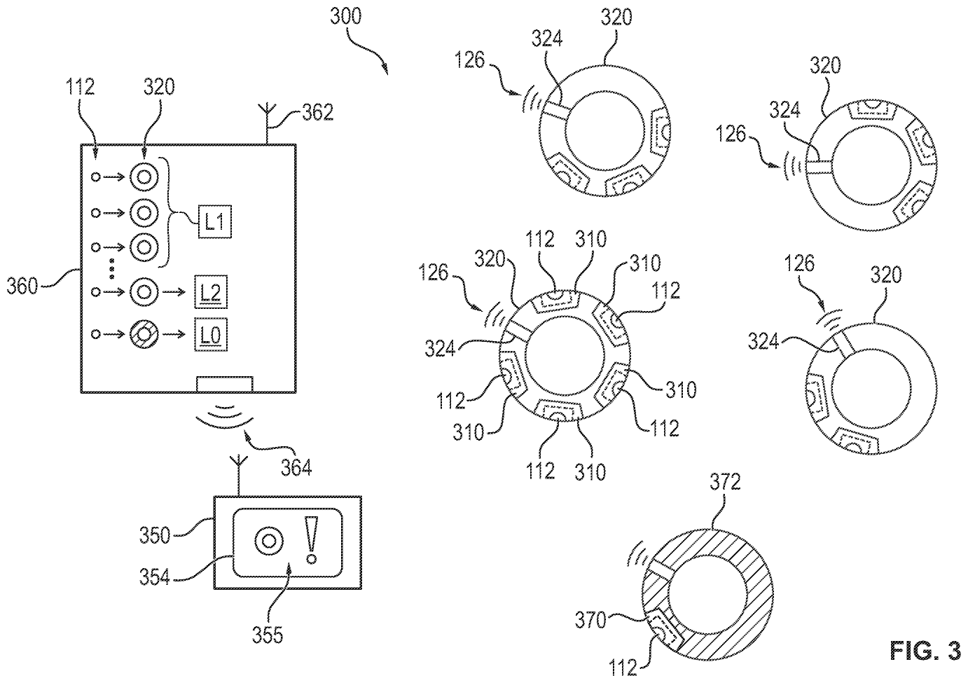

FIG. 3 shows a schematic view of a third exemplary embodiment of the patient management system 300 according to the present invention.

The patient management system 300 shown differs from the patient management systems 100 shown in FIGS. 1 and 2 in that the patient management system 300 comprises, furthermore, a patient monitoring device 360.

The patient monitoring device 360 is configured to detect and to store an assignment between the patient-specific information 112 and the mobile user carrier device 320 with the corresponding patient identifier 310 based on the output signal 126 of the communication interface 324 of the mobile user carrier device 320. The output signal 126 is received in this case by an antenna module 362.

Four patient identifiers 310 are shown here as an example, which output a corresponding output signal 126 to the patient monitoring device 360. The patient-specific information 112 is outputted in this case with a respective output signal 126 for each of the patient identifiers 310 present. As an alternative or in addition, separate output signals are outputted for each patient identifier in one exemplary embodiment, not shown. The mobile user carrier devices 320 have between two and five patient identifiers 310 in the exemplary embodiment being shown. These are brought, especially clicked in, onto the ring-shaped mobile user carrier device 320 via a positive-locking and/or non-positives connection.

Furthermore, a patient-specific visitor identifier 370 is shown in the exemplary embodiment of the patient management system 300. The patient-specific information 112 of the patient assigned to the visitor identifier 370 is assigned to the patient-specific visitor identifier 370 and stored in an internal memory of this visitor identifier. The mobile user carrier device 372 of the visitor identifier 370 can be optically distinguished from the mobile user carrier devices 320 used by medical professional staff on the basis of its dark color.

The patient monitoring device 360 is further configured to assign a respective authorization level, in this case L0, L1, L2, to the respective mobile user carrier device 320 on the basis of the known predefined user of the mobile user carrier device 320. Here, L0 designates the authorization level of a visitor. The visitor is allowed, for example, to open the doors that lead to the patient assigned to the visitor. However, no treatment output and also no alarm output are forwarded to the visitor. As an alternative, the visitor can receive an alarm output so that he can be informed of acute changes in the health status of the patient assigned to the visitor. The authorization level L1 is the authorization level of nursing staff and the authorization level L2 is the authorization level of a medical specialist, who is also allowed, for example, to adjust critical parameters of a medical device. The communication of the patient monitoring device 360 with additional external devices is preferably always based on the authorization which is assigned to the mobile user carrier device 320 of the relevant patient identifier 310.

Finally, the patient monitoring device 360 is further configured to trigger a disconnection output if the communication channel with one of the mobile user carrier devices 320 is disconnected. However, this takes place in this case only when the corresponding mobile user carrier device 320 is assigned to a patient identifier 310 being monitored. The disconnection output 364 is received in this case by an external output device 350, which triggers via a display 354 a visual output 355, which illustrates that an alarm is present for a specific mobile user carrier device 320. The disconnection output 364 is preferably triggered only when the communication channel was disconnected for a predefined time period, especially when the communication channel is disconnected from the corresponding mobile user carrier device 320 for at least 30 sec, preferably for at least 2 minutes, and especially preferably for at least 4 minutes. It is avoided thereby that a temporary interruption of the communication channel based on signal fluctuations or the like would lead to the output of an alarm at the external output device 350. This advantageously leads to an avoidance of manual management effort. The external output device 350 may be provided in this exemplary embodiment in a central management center of the hospital.

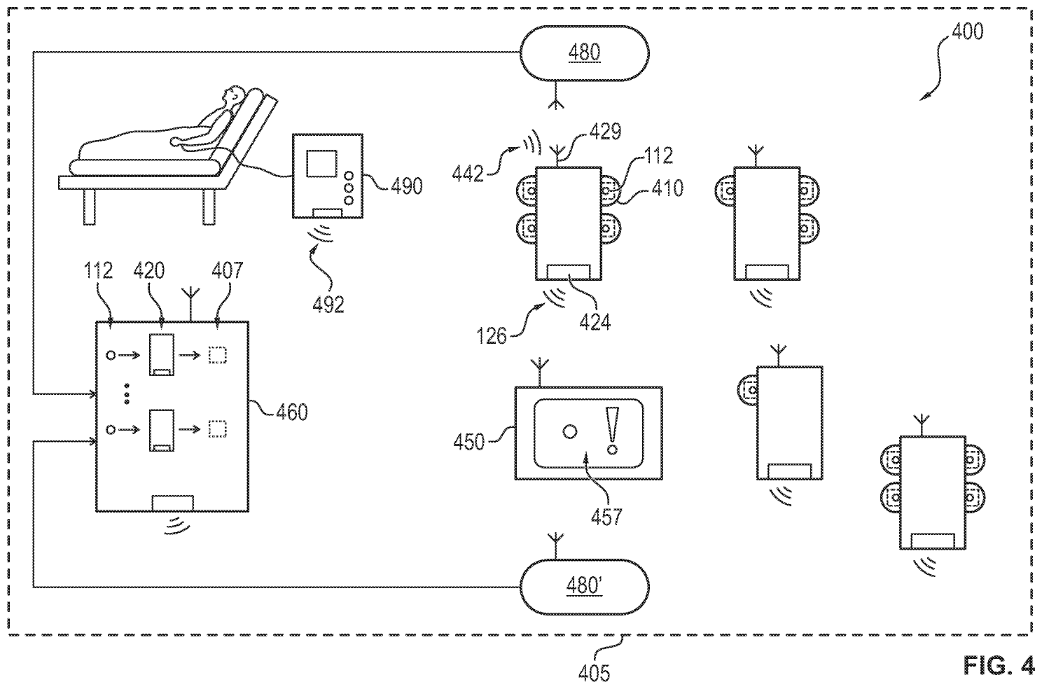

FIG. 4 shows a schematic view of a fourth exemplary embodiment of the patient management system 400 according to the present invention.

The patient management system 400 differs from the patient management system 300 in that it has, furthermore, two locating units 480, which are connected to the patient monitoring device 460 and which are configured to determine the position 407 of at least one mobile user carrier device 420 in a region (building or campus) 405. The patient monitoring device 460 assigns this determined position 407 to the mobile user carrier device 420 or to the patient-specific information 112. As a result, it is determined in an automated manner where the person in charge of a corresponding patient, who has the corresponding mobile user carrier device 420, is located in the building 405. The position determination may be based, for example, on an analysis of a signal intensity of the output signal 126 outputted by the mobile user carrier device 420. In an alternative or additional embodiment, at least one additional locating units is provided, via which the position of the mobile user carrier device in the building is determined, doing so especially by triangulation. Such processes for the position determination are generally known and will not therefore be explained in detail below.

Due to the assigned position 407 in the building 405, the patient monitoring device 460 is configured to output information for the user of the mobile user carrier device 420, e.g., the management signal shown within the framework of FIG. 2, to an external output device 450 in a space surrounding the assigned position 407 of the mobile user carrier device 420 and to trigger thereby a device output, especially an alarm output, or a treatment output, by this external output device 450. Thus, a position of the mobile user carrier device 420 assigned to a patient identifier 410 can be determined in this exemplary embodiment by means of the locating units 480, 480′, so that a device output 457 for the user of this patient identifier 410 takes place at an external output device 450 located in spatial proximity. The device output 457 at the external output device 450 is preferably information that is assigned to the patient identifier 410 and pertains to the patient, e.g., vital information, a treatment recommendation, a device error, an alarm or the like.

Finally, the patient monitoring device 460 is further configured in the exemplary embodiment shown to receive a forwarded signal 492 assigned to the patient identifier 410, especially from a medical device 490 intended for monitoring the corresponding patient. This signal is outputted here directly as a patient signal 442 to the mobile user carrier device 420 assigned to the patient identifier 410. For example, an output, which outputs to the user of the mobile user carrier device 420 information originally indicated by the forwarded signal 492, can take place hereby directly at the mobile user carrier device 420.

To receive the patient signal 494, each mobile user carrier device 420 has in the exemplary embodiment shown an antenna module 429 in addition to the communication interface 424. The entire communication takes place via the communication interface in an alternative exemplary embodiment.

The patient monitoring device is configured in an alternative or additional exemplary embodiment to communicate with an alarm management system in order to receive an alarm from this and to output same to the external output device corresponding to the position of the mobile user carrier device that is relevant according to the assignment being stored. Corresponding alarm information is preferably forwarded in this connection.

The storage of the patient identifiers is carried out in the exemplar embodiment shown by respective identifier receptacles at the lateral edge of the mobile user carrier device 420. The identifier receptacles are configured in this case such that the respective identifier receptacles project beyond the respective patient identifiers over the edge of the record and can be detected thereby by an outsider when looking at the mobile user carrier device 420. The number of patient identifiers 410 assigned to the mobile user carrier device 420 can be determined thereby optically rapidly and reliably.

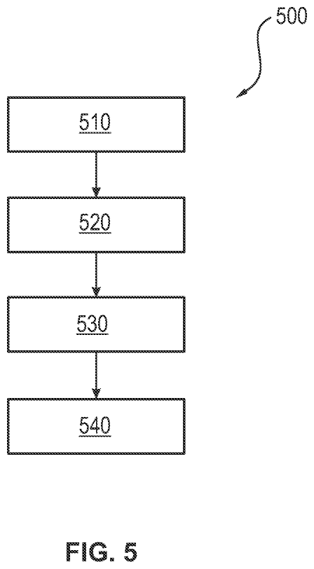

FIG. 5 shows a flow chart of an exemplary embodiment of a process 500 according to another aspect of the present invention.

The process 500 according to the present invention is configured for managing an assignment to a patient. It has for this purpose the steps described below.

A first step 510 comprises a storage of patient-specific information on a patient identifier.

A next step 520 comprises a storage of a number of patient identifiers at a mobile user carrier device, so that a number of patient identifiers currently being stored at the mobile user carrier device can be viewed (visually recognized-optically detected), the mobile user carrier device having an energy storage device and a communication interface.

A next step 530 comprises a reading out of the respective patient-specific information of the patient identifiers stored at the mobile user carrier device by the mobile user carrier device.

A further step 540 comprises a wireless output of the read-out patient-specific information via the communication interface.

Steps 510, 520, 530 and 540 are carried out typically in the order shown. The storage of the patient-specific information on the patient identifier may take place at different times relative to the further process steps. Thus, the patient-specific information may be stored permanently on the patient identifier, for example, by a bar code, a QR code, an RFID tag or the like. The patient-specific information may be a fixed character string, which characterizes, for example, a specific (certain physical) bed location or the specific (certain physical) patient identifier. The patient-specific information is patient-specific here in the respect that this specific information, e.g., the specific bed location or the specific patient identification is unambiguously assigned to the patient at the beginning of his treatment, so that this information is stored specifically for this patient and it thus allows an unambiguous assignment between patient-specific information and patient. As an alternative or in addition, step 510 may take place at the beginning of the treatment of a patient, for example, at the time of his admission in a hospital. The patient-specific information is in this case a name, a date of birth or similar characterizing information of the patient.

Step 520 may be carried out several times after step 510 has been carried out for a single time, for example, when a patient identifier is moved at the time of a change of shifts from one mobile user carrier device, which is assigned to a nursing staff (medical staff) member, to another mobile user carrier device of another nursing staff (medical staff) member.

Steps 530 and 540 are carried out typically immediately after step 520, because the patient-specific information is read out and outputted after the patient identifier has been arranged at a mobile user carrier device. Preferably less than 10 minutes, especially less than 5 minutes and especially preferably less than 1 minute pass between the arrangement of the patient identifier and the output of at least some of the assigned patient-specific information.

The process 500 according to the present invention may be supplemented by one or more additional process steps in advantageous exemplary embodiments. Thus, the process according to the present invention may additionally comprise, for example, the step of receiving a patient signal and of a subsequent triggering of an output of the mobile user carrier device and/or of the corresponding patient identifier. The output may be, for example, an alarm output or a treatment output.

As an alternative or in addition, the process according to the present invention may additionally also comprise the process step of detecting and storing an assignment between the patient-specific information and the mobile user carrier device based on the output of the mobile user carrier device.

The communication between the patient identifier and the mobile device and between the mobile device and possible additional devices preferably takes place wirelessly, because the mobile user carrier device shall be free from cables to external devices and the patient identifier communicated with the mobile user carrier device wirelessly. The communication between the patient identifier and the mobile user carrier device may, however, also take place via a corresponding socket at the mobile user carrier device in a cable-based manner.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 300, 400 Patient management system
110, 210, 310, 410 Patient identifier
112 Patient-specific information
114 Internal memory
116 Identifier interface
120, 220, 320, 420 Mobile user carrier device
121 Identifier record
122 Energy storage device
124, 324, 424 Communication interface
125, 225 Receiving unit
126 Output signal
130 Clip
221 Magnetic surface
228 Output
240 Managing device
242, 442 Patient signal
250, 350, 450 External output device
252 Antenna unit
254, 354 Display
255, 355 Visual output
256 Speaker
257, 457 Device output
360, 460 Patient monitoring device
362 Antenna module
364 Disconnection output
370 Visitor identifier
372 Mobile user carrier device of the visitor identifier
405 Building
407 Position
480, 480′ Locating unit
429 Antenna module of the mobile user carrier device
490 Medical device
492 Forwarded signal
500 Process
510, 520, 530, 540 Process steps

What is claimed is:

1. A patient management system for managing an assignment to a patient, the patient management system comprising:
a plurality of patient identifiers, wherein the plurality of patient identifiers are intended for a medical staff and each of the plurality of patient identifiers has patient-specific information stored thereon, and each of the plurality of patient identifiers is assigned to a corresponding one of the plurality of patients;
a mobile user carrier device configured to store one or more of the plurality of patient identifiers, such that a number of patient identifiers being stored at the mobile user carrier device is optically detectable, wherein:
the mobile user carrier device comprises an energy storage device;
the mobile user carrier device comprises a communication interface; and
the mobile user carrier device is configured to read out the respective patient-specific information of the patient identifiers being stored at the mobile user carrier device at least partially and to output same wirelessly via the communication interface.

2. A patient management system in accordance with claim 1, wherein each patient identifier, of the plurality of patient identifiers, comprises an internal memory, which is configured to store the patient-specific information at least for a patient treatment duration.

3. A patient management system in accordance with claim 1, wherein each patient identifier, of the plurality of patient identifiers, comprises an identifier interface, which is configured for a wireless transmission of the patient-specific information.

4. A patient management system in accordance with claim 3, wherein the identifier interface is a near-field communication interface or a radio-frequency identification interface for the wireless transmission.

5. A patient management system in accordance with claim 1, wherein the mobile user carrier device has a plurality of identifier receptacles, via which the number of patient identifiers currently being stored at the mobile user carrier device are viewably stored.

6. A patient management system in accordance with claim 1, wherein the mobile user carrier device is configured to receive, from a managing device, a patient signal assigned to the patient identifier and to trigger a corresponding output of the mobile user carrier device or trigger a corresponding output of the corresponding patient identifier or trigger a corresponding output of both the mobile user carrier device and the corresponding patient identifier.

7. A patient management system in accordance with claim 6, wherein the output comprises at least one of a tactile output, an optical output and an acoustic output.

8. A patient management system in accordance with claim 6, wherein the corresponding output of the mobile user carrier device or the corresponding output of the patient identifier or corresponding output of both the mobile user carrier device and the corresponding patient identifier comprises a wireless output to an external output device, located in a predefined surrounding space, to trigger an output by the external output device.

9. A patient management system in accordance with claim 1, further comprising a patient monitoring device configured to detect and to store an assignment between the patient-specific information and the mobile user carrier device with the corresponding patient identifier based on the output of the communication interface of the mobile user carrier device.

10. A patient management system in accordance with claim 9, wherein the patient monitoring device is further configured:
to assign to the mobile user carrier device an authorization level; and
to carry out a communication, which is triggered by a patient identifier assigned to the mobile user carrier device, based on the assigned authorization level.

11. A patient management system in accordance with claim 9, further comprising at least two locating units configured to determine a position of the mobile user carrier device in a region, wherein the patient monitoring device is configured to assign the correspondingly determined position in the region to the mobile user carrier device or to patient-specific information of one or more of the number of patient identifiers being stored at the mobile user carrier device.

12. A patient management system in accordance with claim 11, wherein the patient monitoring device is configured to trigger a device output at an external output device, located in a predefined surrounding space, wherein the output is in dependence on the assigned position being associated with the predefined surrounding space.

13. A patient management system in accordance with claim 9, wherein:
the mobile user carrier device is configured to receive, from a managing device, a patient signal assigned to the patient identifier and to trigger a corresponding output of the mobile user carrier device or trigger a corresponding output of the corresponding patient identifier or trigger a corresponding output of both the mobile user carrier device and the corresponding patient identifier; and
the patient monitoring device is further configured to receive a forwarded signal assigned to the patient identifier and to output same as a patient signal to the mobile user carrier device.

14. A patient management system in accordance with claim 9, wherein the patient monitoring device is further configured to trigger a disconnection output if a communication channel is disconnected from the mobile user carrier device and if this mobile user carrier device is assigned to a monitored patient identifier.

15. A patient management system in accordance with claim 1, further comprising a plurality of patient-specific visitor identifiers, wherein the patient-specific information of the patient assigned to the visitor identifier is stored in each of the patient-specific visitor identifiers.

16. A patient management system in accordance with claim 1, wherein the mobile user carrier device is configured as a necklace, a bracelet or a clip.

17. A patient management system in accordance with claim 1, wherein a respective patient identifier, of the plurality of patient identifiers, is configured to provide an unambiguous optical identification of the patient assigned to the respective patient identifier based on one or more of a color, a symbol, a character string, indicia, and markings of the respective patient identifier.

18. A process for managing an assignment to a patient, the process comprising the steps of:
storing patient-specific information on a patient identifier;
storing a number of patient identifiers at a mobile user carrier device, wherein the number of patient identifiers being stored at the mobile user carrier device is optically detectable, wherein the mobile user carrier device has an energy storage device and a communication interface;
reading out of the respective patient-specific information of the patient identifiers being stored at the mobile user carrier device by the mobile user carrier device; and
wirelessly outputting of the read-out patient-specific information via the communication interface.
